(19) United States
(12) Patent Application Publication
(10) Pub. No.: US 2007/0252016 A1
(43) Pub. Date: Nov. 1, 2007

(54) Perfume device with changeable appearance

(76) Inventor: Shih H. Chen, Yongkang City (TW)

Correspondence Address: HDSL, 4331 STEVENS BATTLE LANE, FAIRFAX, VA, 22033, US

(21) Appl. No.: 11/413,099

(22) Filed: Apr. 28, 2006

(51) Int. Cl. A61L 9/04; A24F 25/00

(52) U.S. Cl. 239/36

(57) ABSTRACT

A perfume device with changeable appearance includes a casing and a decoration plate. A coupling section is arranged on one side of the casing and the casing includes an accommodation space. The accommodation space is used for placement of essence. The casing includes a plurality of air outlets. The decoration plate is replaceably connected to the casing and includes a connection part on backside thereof and pattern on front side thereof. The connection part is fit with the coupling section. Therefore, user can replace decoration plate with pattern fit with odor of essence to achieve visual display effect.

### BACKGROUND OF THE INVENTION

1. Field of the Invention

The present invention relates to a perfume device with changeable appearance, especially to a perfume device for wear on use body or cloth.

2. Description of Prior Art

Modern people are annoyed by high work pressure and they suffer to diseases of nervous system, respiratory system, digest system and internal secretion system. Therefore, aromatherapy with essence is popular for modern people. The essence is made of natural herb and the essence is diffused to air trough conveying unit. The diffused essence can be inhaled by user for relaxation and health-enhancement.

Taiwan patent application filing No. 92115146 discloses a prior art perfume device. The perfume device includes a base, a first cover, a second cover, a PCB and an oscillation unit. The base includes front groove and rear groove, where an oil absorber is placed in the rear groove. The first cover and the second cover are placed at front and rear ends of the base. The PCB is fixed to the front groove. The oscillation unit is arranged on the PCB and connected to circuits on the PCB.

However, the shown prior art perfume device has dull shape without flexibility. Moreover, the shape of the perfume device cannot be changed with respect to oil of different smells. Therefore, it is not attractive to user.

### SUMMARY OF THE INVENTION

The present invention is to provide a perfume device with changeable appearance, whereby user can replace decoration plate with pattern fit with odor of essence to achieve visual display effect.

Accordingly, the present invention provides a perfume device with changeable appearance. The perfume device is for wear on user body and includes a casing and a decoration plate. A coupling section is arranged on one side of the casing and the casing includes an accommodation space. The accommodation space is used for placement of essence. The casing includes a plurality of air outlets. The decoration plate is replaceably connected to the casing and includes a connection part on backside thereof and pattern on front side thereof. The connection part is fit with the coupling section.

### BRIEF DESCRIPTION OF DRAWING

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 shows the exploded view of the present invention.

FIG. 2 shows the perspective of the present invention.

FIG. 3 shows the exploded view of the casing of present invention.

FIG. 4 is a lateral sectional view of the present invention.

FIG. 5 is a longitudinal sectional view of the present invention.

FIG. 6 is a schematic view of another preferred embodiment of the present invention.

FIG. 7 is an exploded view of another preferred embodiment of the present invention.

FIG. 8 is a schematic view of still another preferred embodiment of the present invention.

### DETAILED DESCRIPTION OF THE INVENTION

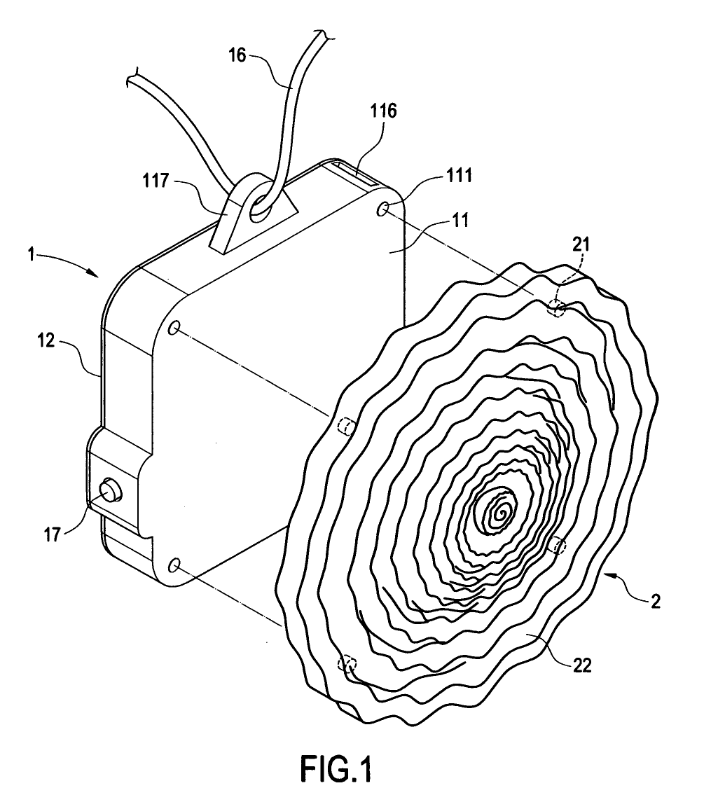

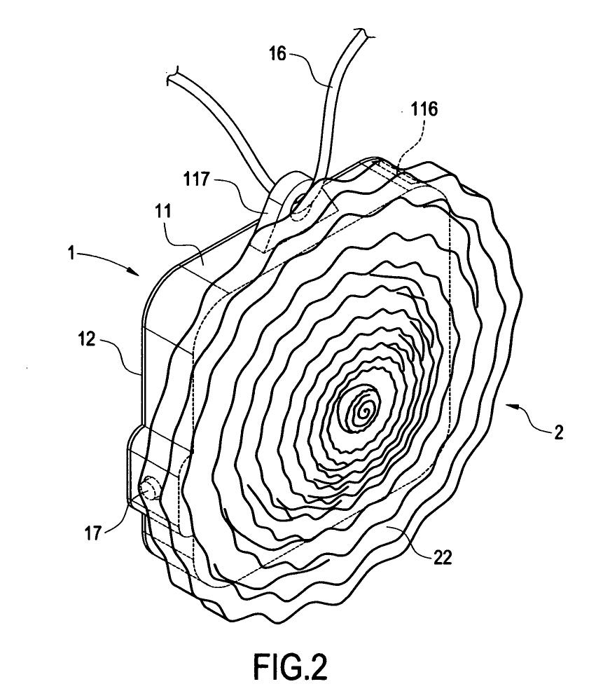

With reference to FIGS. 1 to 3, the perfume device with changeable appearance according to the present invention includes a casing 1 and a decoration plate 2.

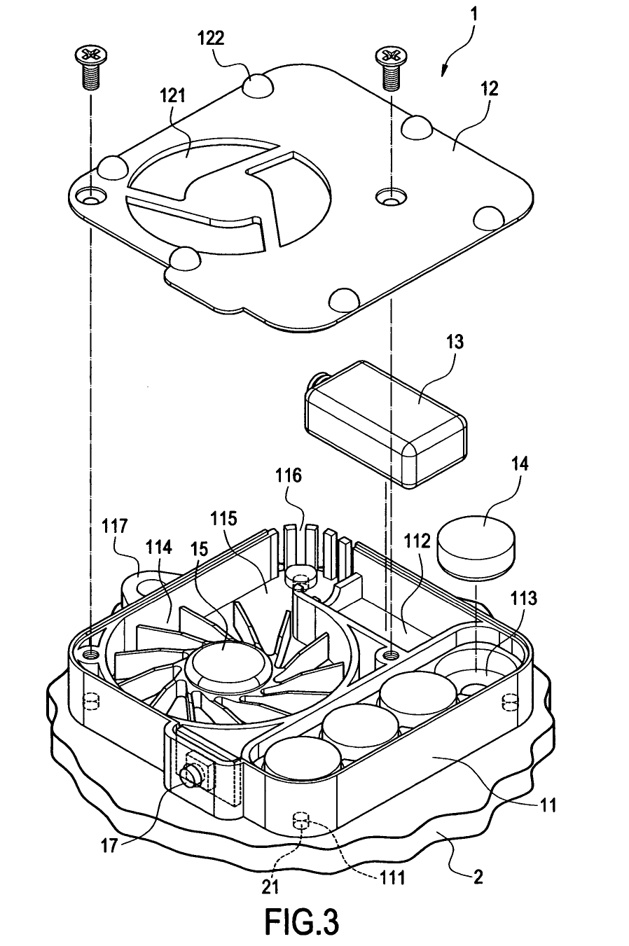

The casing 1 is of rectangular shape. With reference to FIG. 3, the casing 1 includes a box 11 and a cover plate 12 atop the box 11. A coupling section 111 is provided at bottom of the box 11. In the preferred embodiment, the coupling section 111 is a concave hole. An accommodation space 112 for essence, a battery room 13 and a fan tank 114 are defined in the box 11. The accommodation space 112 is provided for the placement of essence 13, which can be liquid oil placed in a bottle.

The battery room 13 is provided for the placement of at least one battery and the fan tank 114 is provided for the placement of an electrical fan 15. A wind conduit 115 is provided corresponding to one side of the accommodation space 112 and a plurality of air outlets 116 are defined at lateral side of the box 11. As shown in FIG. 5, the exit opening for the essence 13 faces the wind conduit 115 and the perfume of the essence 13 flows out of the air outlets 116. A fixing rack 117 is extended from one lateral side of the box 11 and a hanging strip 16 passing through the fixing rack 117. User can hang the perfume device on his neck by the hanging strip 16. Moreover, bar-shaped wind inlet 121 is defined at the cover plate 12 and corresponding to the fan tank 114. A plurality of projections 122 is formed at outer face of the cover plate 12.

The decoration plate 2 is replaceably arranged on the box 11 and the decoration plate 2 comprises a connection part 21 at backside and a pattern 22 at front side thereof. In this preferred embodiment, the connection part 21 is a post and corresponding to the coupling section 111. The pattern 22 can have various graphs and shapes such as rose, tulip and lavender. Moreover the decoration plate 2 has a larger area than that of the casing 1 to cover the casing 1.

Moreover, the perfume device further comprises a control switch 17 arranged on the box 11 and electrically connected to the electrical fan 15 to control the on/off of the electrical fan 15.

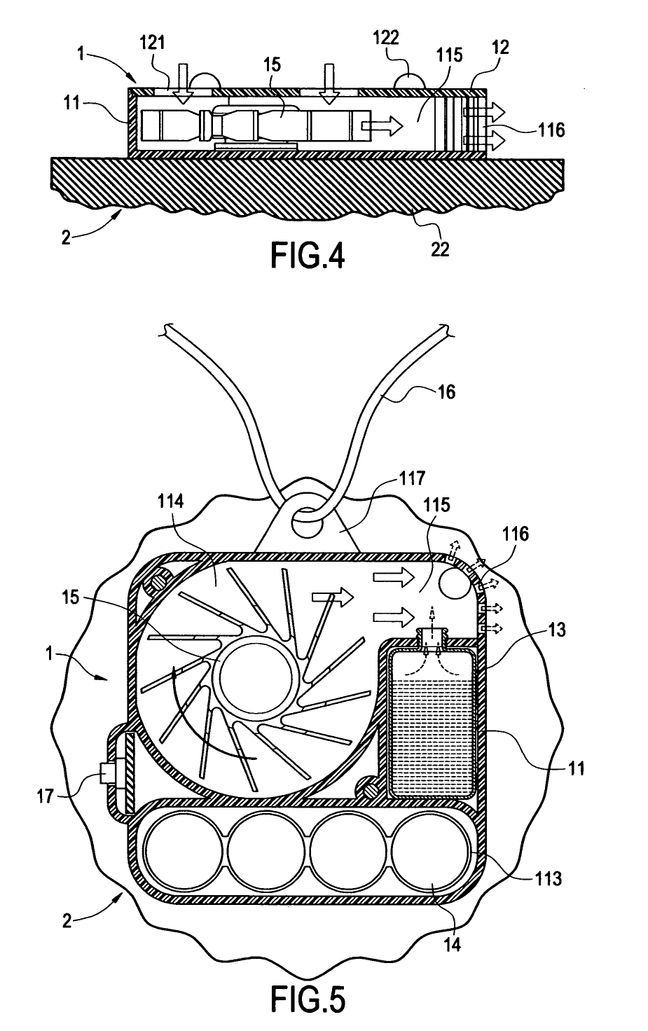

With reference to FIGS. 4 and 5, during use of the perfume device, the perfume device can be hang on the neck of user by the hanging strip 16. The control switch 17 is operated to rotate the electrical fan 15 such that air is drawn into the casing 1 through the wind inlet 121 and corresponding to the opening of the essence 13 projecting in the wind conduit 115. Therefore, the odor of the essence 13 can be forced to eject from the air outlets 116.

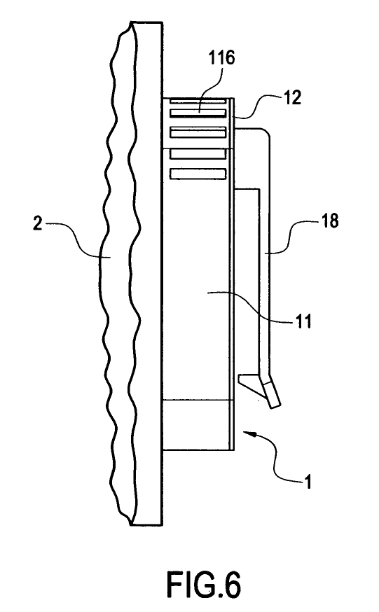

FIG. 6 shows another preferred embodiment of the present invention. The perfume device according to the present invention further comprises a clamping arm 18, which can be clamped to cloth of user to function as pectoral decoration.

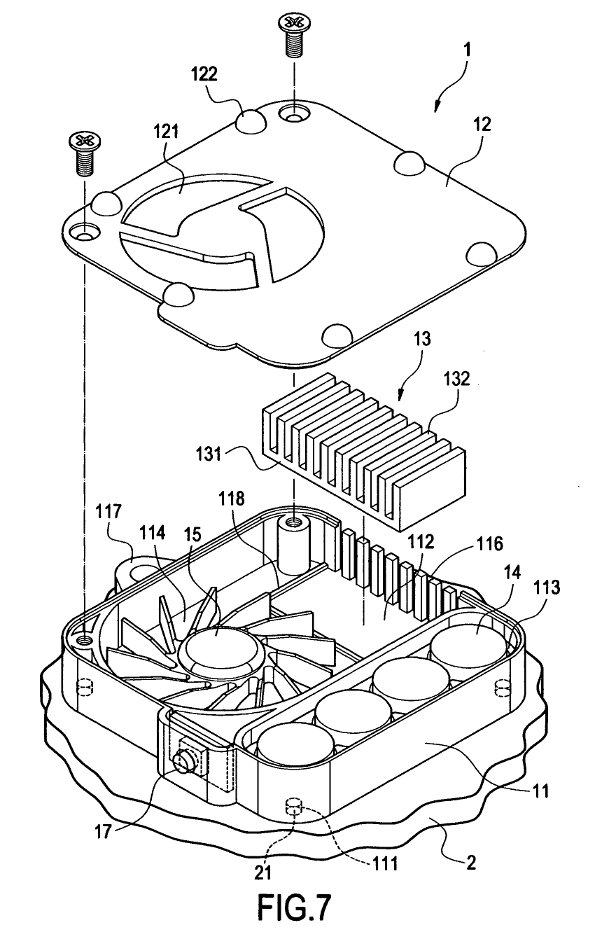

FIG. 7 shows another preferred embodiment of the present invention. An L-shaped wall 118 is projected on bottom face of the box 11. The accommodation space 112 is defined beside he wall 118 and sidewall of the box 11. The accommodation space 112 can be used to place a solid-state essence 13. The solid-state essence 13 comprises a panel 131 and a plurality of plates 132 extended from one side of the panel 131 such that the solid-state essence 13 has larger surface area. When the solid-state essence 13 is blown by the electrical fan 15, the perfume dissipation effect can be enhanced.

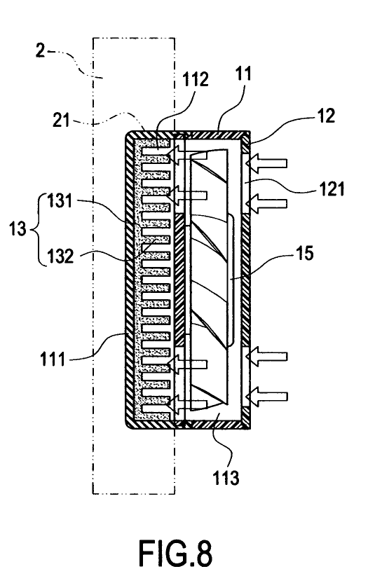

FIG. 8 shows another preferred embodiment of the present invention. The connection part 21 of the decoration plate 2 is a groove and the coupling section 111 of the box 11 is a frame, where the accommodation space 112 is arranged in the frame such that the decoration plate 2 can be directly arranged outside the frame. A maximal accommodation space 112 can be obtained without changing the length and width of the casing 1. Therefore, a large-size solid-state essence 13 can be placed in the accommodation space 112 with prolonged time.

Although the present invention has been described with reference to the preferred embodiment thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the appended claims.

What is claimed is:

1. A perfume device with changeable appearance, comprising
a casing having a coupling section and an accommodation space therein and used for placement of an essence, a plurality of air outlets being defined on the casing; and
a decoration plate replaceably arranged on one side of the casing, the decoration plate comprising a connection part on back side and a pattern on front side, the connection part being fit with the coupling section.

2. The perfume device with changeable appearance as in claim 1, wherein the coupling section is a concave hole and the connection part of the decoration plate is a post.

3. The perfume device with changeable appearance as in claim 1, wherein the casing comprises a box and a cover plate corresponding to the box, the box containing a battery room and a fan tank, the battery room being used for placement of a battery and the fan tank being used for placement of an electrical fan.

4. The perfume device with changeable appearance as in claim 3, wherein the fan tank comprises a row of wind conduits and the wind conduits are arranged on one side of the accommodation space such that one end of the essence is projected out of the wind conduit.

5. The perfume device with changeable appearance as in claim 3, wherein the cover plate comprises wind inlets corresponding to one side of the fan tank, a plurality of projections is formed on outer surface of the cover plate.

6. The perfume device with changeable appearance as in claim 3, wherein the accommodation space is defined on a bottom plate of the box and corresponding to a front face of the electrical fan.

7. The perfume device with changeable appearance as in claim 3, wherein a wall is projected from bottom side of the box and the accommodation space is defined between the wall and sidewall of the box.

8. The perfume device with changeable appearance as in claim 3, further comprising a control switch on the box and electrically connected to the electrical fan for turning on/off the electrical fan.

9. The perfume device with changeable appearance as in claim 1, wherein the essence is liquid.

10. The perfume device with changeable appearance as in claim 1, wherein the essence is solid.

11. The perfume device with changeable appearance as in claim 10, wherein the solid-state essence comprises a panel and a plurality of plate extended upward from one side of the panel.

12. The perfume device with changeable appearance as in claim 1, wherein the casing further comprises a fixing stage passed by a hanging strip.

13. The perfume device with changeable appearance as in claim 1, wherein the casing further comprises a clamping arm for clamping to cloth.

14. The perfume device with changeable appearance as in claim 1, wherein the connection part of the decoration plate is a groove and the coupling section of the box is a frame.

15. The perfume device with changeable appearance as in claim 1, wherein the area of the decoration plate is larger than that of the casing.
